(19) United States
(12) Patent Application Publication
(10) Pub. No.: US 2013/0149774 A1
(43) Pub. Date: Jun. 13, 2013

(54) COLLECTIVE CELL COUNTER SYSTEM

(71) Applicants: Min-seok KIM, Busan (KR); Tae-seok SIM, Seoul (KR); Yeon-jeong KIM, Yongin-si (KR); Sang-hyun BAEK, Hwaseong-si (KR); Jin-mi OH, Suwon-si (KR); Hyo-young JEONG, Suwon-si (KR); Jeong-gun LEE, Seoul (KR)

(72) Inventors: Min-seok KIM, Busan (KR); Tae-seok SIM, Seoul (KR); Yeon-jeong KIM, Yongin-si (KR); Sang-hyun BAEK, Hwaseong-si (KR); Jin-mi OH, Suwon-si (KR); Hyo-young JEONG, Suwon-si (KR); Jeong-gun LEE, Seoul (KR)

(73) Assignee: SAMSUNG ELECTRONICS CO., LTD., Suwon-si (KR)

(21) Appl. No.: 13/615,267

(22) Filed: Sep. 13, 2012

(30) Foreign Application Priority Data
Dec. 12, 2011 (KR) 10-2011-0133056

(51) Int. Cl. G01N 27/02; C12M 1/36

(52) U.S. Cl. 435/286.2; 324/692

(57) ABSTRACT

A cell counter system includes an inlet via which a fluid containing a plurality of cells inflows; a channel in which the fluid flows; a valve unit, which controls flow of the fluid in the channel; an electrode unit, which is arranged in the channel for measuring impedance for counting a number of the plurality of cells; and an outlet, which is connected to the channel to drain the fluid.

### CROSS-REFERENCE TO RELATED APPLICATIONS

This application claims the benefit of Korean Patent Application No. 10-2011-0133056, filed on Dec. 12, 2011, in the Korean Intellectual Property Office, the disclosure of which is incorporated herein in its entirety by reference.

### BACKGROUND

Microfluidic devices refer to devices for performing biological or chemical reaction by manipulating small amount of fluids. A microfluidic device includes microfluidic structures arranged in various types of platforms, such as chips and disks, and includes a chamber for storing a fluid, a channel in which a fluid may flow, and a valve for controlling flow of a fluid, for example.

Recently, a microfluidic platform-based circulating tumor cell (CTC) diagnosis chip has been developed. It has been confirmed that CTCs may be effectively separated from blood of a patient by using the CTC diagnosis chip. A CTC is a tumor cell circulating in blood and found in peripheral blood of a cancer patient, where it is thought that the CTC causes metastasis of cancer. Such a new technology is an innovative technology that may be used for diagnosing and detecting cancer. However, it is known as very difficult to separate such cells from a cancer patient. The main reason is that only a very small number of such cells exist in peripheral blood.

Currently, no equipment capable of counting a number of cells of a concentration below or equal to about 10⁴ cells/mL has been commercialized. Furthermore, no system that is not only capable of counting cells, but also capable of being collected has been reported. A technique for spiking an accurate number of cells may reduce experimental errors in CTC analysis, enable measurement of an accurate collection ratio, and reduce massive amount of effort and time for spiking. Therefore, the technique is expected to improve efficiency of CTC analysis and development.

### SUMMARY

Provided are methods and apparatuses for a collective cell counter system.

Additional aspects will be set forth in part in the description which follows and, in part, will be apparent from the description, or may be learned by practice of the presented embodiments.

According to an aspect of the present invention, a cell counter system includes an inlet via which a fluid containing a plurality of cells inflows; a channel in which the fluid flows; a valve unit, which controls flow of the fluid in the channel; an electrode unit, which is arranged in the channel for measuring impedance for counting a number of the plurality of cells; and an outlet, which is connected to the channel to drain the fluid.

The cell counter system may further include a control unit, which counts a number of cells based on the measured impedance and controls operation of the valve unit.

The channel may be formed by a first substrate and a second substrate, which face each other and are a predetermined distance apart from each other, and a spacer, which seals a space between the first substrate and the second substrate in a predetermined shape. The predetermined shape may include a shape tapered from the inlet to the outlet.

A via hole for forming the inlet and a via hole for forming the outlet may be formed in the second substrate.

The valve unit may include a valve film, which is a portion of the second substrate worked to have a relatively small thickness; and a valve rod, which presses the top surface of the valve film, such that the valve film contacts the bottom surface of the channel.

A first groove having a predetermined depth may be formed on the second substrate, and the bottom surface of the first groove may become the top surface of the valve film. A second groove may be formed on a surface of the second substrate opposite to the surface on which the first groove is formed.

The cell counter system may further include a mounting unit, on which mounting grooves for mounting the first substrate and the second substrate are formed; and a cover unit, which is hinge-attached to the mounting unit for opening and closing the mounting grooves and includes an actuator for driving the valve rod and a pumping unit for introducing a fluid via the inlet.

The electrode unit may include a first electrode and a second electrode that are apart from each other in a direction in which the fluid flows. The electrode unit may be arranged on the first substrate.

The cell counter system may further include an auxiliary channel, which splits at a portion of the channel close to the inlet and is recombined at a portion of the channel close to the outlet and has a width smaller than that of a cell contained in the fluid.

The channel and the auxiliary channel may be formed by a first substrate and a second substrate, which face each other and are a predetermined distance apart from each other, and a spacer, which seals a space between the first substrate and the second substrate in a predetermined shape.

The channel may be formed to split into two directions at a predetermined location past the electrode unit in a direction in which the fluid flows. In this case, the outlet may include a first outlet and a second outlet that are respectively formed at ends of the channel split into two directions.

A via hole for forming the inlet, a via hole for forming the first outlet, and a via hole for forming the second outlet may be formed in the second substrate.

A first auxiliary valve unit for controlling flow of a fluid may be further provided between the predetermined location and the first outlet, and a second auxiliary valve unit for controlling flow of a fluid may be further provided between the predetermined location and the second outlet.

The cell counter system may further include a control unit, which categorizes cells to first cells and second cells based on the measured impedances and controls operation of the first auxiliary valve unit and the second auxiliary valve unit, such that the first cells move toward the first outlet and the second cells move toward the second outlet.

Each of the first and second auxiliary valve unit may include a valve film, which is a portion of the second substrate worked to have a relatively small thickness; and a valve rod, which presses the top surface of the valve film, such that the valve film contacts the bottom surface of the channel.

Grooves having a predetermined depth for forming each of the valve unit, the first auxiliary valve unit, and the second auxiliary valve unit may be formed on the second substrate.

The cell counter system may further include a mounting unit, on which mounting grooves for mounting the first substrate and the second substrate are formed; and a cover unit, which is hinge-attached to the mounting unit for opening and closing the mounting grooves and includes an actuator for driving a valve rod and a pumping unit for introducing a fluid via an inlet.

### BRIEF DESCRIPTION OF THE DRAWINGS

These and/or other aspects will become apparent and more readily appreciated from the following description of the embodiments, taken in conjunction with the accompanying drawings of which:

FIG. 1 is a schematic perspective view of a cell counter system according to an embodiment of the present invention;

FIG. 2 is a sectional view of the cell counter system, taken along a line A-A′ of FIG. 1;

FIGS. 3A through 3C show embodiments of a valve unit that may be employed by the cell counter system of FIG. 1;

FIG. 4 is a block diagram for describing operation of the cell counter system of FIG. 1;

FIG. 5 is a schematic perspective view of a cell counter system according to another embodiment of the present invention;

FIG. 6 is a schematic perspective view of a cell counter system according to another embodiment of the present invention;

FIG. 7 is a graph of an experiment showing that signals detected by an electrode unit of the cell counter system may differ according to types of cells contained in a fluid; and

FIG. 8 is a schematic perspective view of a cell counter system according to another embodiment of the present invention.

### DETAILED DESCRIPTION

Reference will now be made in detail to embodiments, examples of which are illustrated in the accompanying drawings, wherein like reference numerals refer to the like elements throughout. In this regard, the present embodiments may have different forms and should not be construed as being limited to the descriptions set forth herein. Accordingly, the embodiments are merely described below, by referring to the figures, to explain aspects of the present description.

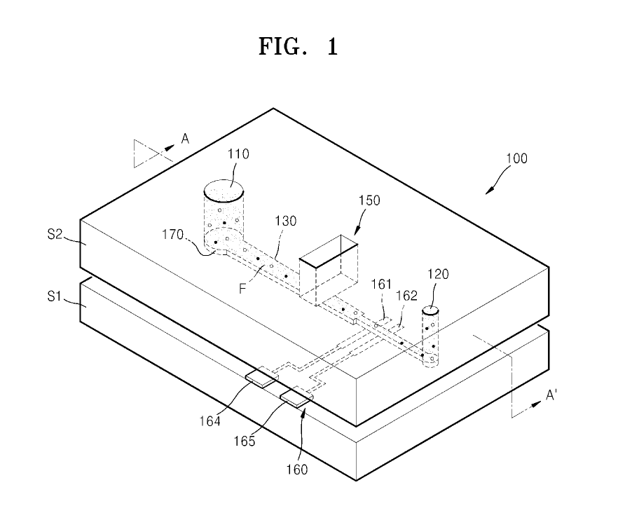

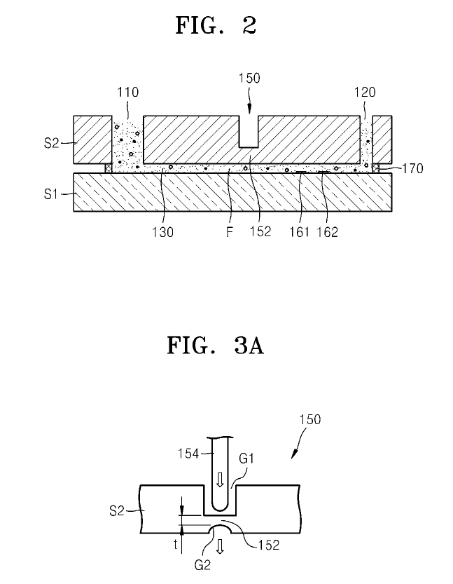

FIG. 1 is a schematic perspective view of a cell counter system 100 according to an embodiment of the present invention, and FIG. 2 is a sectional view of the cell counter system 100, taken along a line A-A′ of FIG. 1.

Referring to FIGS. 1 and 2, the cell counter system 100 includes an inlet 110 via which a fluid F containing a large number of cells inflows, a channel 130 in which the fluid F flows, a valve unit 150 which controls flow of the fluid F in the channel 130, an electrode unit 160 arranged at the channel 130 for measuring impedance for counting a number of plurality of cells, and an outlet 120 which is connected to the channel 130 and via which the fluid F is drained. Furthermore, a control unit for counting cells based on the measured impedance and controlling operation of the valve unit 150 may be further arranged.

As shown in FIGS. 1 and 2, the channel 130 may be formed by a first substrate S1 and a second substrate S2 which are a predetermined distance apart from each other and a spacer 170 which seals the space between the first substrate S1 and the second substrate S2 in a predetermined shape.

At least one of the first substrate S1 and the second substrate S2 may be a transparent substrate. In a case where the first substrate S1 or the second substrate S2 is a transparent substrate, it may be easy to observe the fluid F in the channel 130.

The first substrate S1 may be formed of a glass, a quartz, a plastic, a polymer, etc.

The second substrate S2 is arranged above the first substrate S1 to be a predetermined distance apart from the first substrate S1 and may be formed of silicon, a silicon-based polymer material, or a polymer material, for example. In detail, the second substrate S2 may be formed of acrylate, polymethylacrylate, polymethylmethacrylate (PMMA), polycarbonate, polystyrene, polyimide, epoxy resin, polydimethylsiloxane (PDMS), parylene, etc.

The spacer 170 seals the space between the first substrate S1 and the second substrate S2 in a predetermined shape, thus forming the channel 130. The channel 130 may have a shape tapered from the inlet 110 to the outlet 120 for flow of the fluid F. The spacer 170 may be integrated with the first substrate S1 or the second substrate S2.

The electrode unit 160 is arranged to measure change of impedance according to flow of the fluid F and is arranged on the first substrate S1 across the bottom surface of the channel 130. In detail, the electrode unit 160 includes a first electrode 161 and a second electrode 162 that are arranged across the bottom surface of the channel 130 and are apart from each other in a direction in which the fluid F flows. The first electrode 161 and the second electrode 162 are respectively connected to a first electrode pad 164 and a second electrode pad 165 that are connected to an impedance measuring unit (not shown).

The valve unit 150 includes a valve film 152, which is a portion of the second substrate S2 worked to have a relatively small thickness, and a valve rod, which presses the top surface of the valve film 152, such that the valve film 152 contacts the bottom surface of the channel 130. As shown in FIGS. 1 and 2, a groove having a predetermined depth is formed in the second substrate S2, and the bottom surface of the groove becomes the top surface of the valve film 152. The valve rod is moved up and down by a valve driving unit, such as an actuator, and deforms the valve film 152.

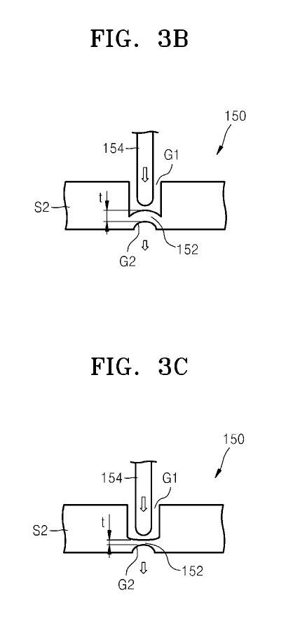

FIGS. 3A through 3C show embodiments of the valve unit 150 that may be employed by the cell counter system 100 of FIG. 1.

Referring to FIG. 3A, the valve film 152 may be formed to have a thickness t due to a groove G1 formed on the top surface of the second substrate S2 and a groove G2 formed on the rear surface of the second substrate S2. As the valve rod 154 moves up and down, the valve film 152 is deformed upward and downward, and thus flow of a fluid is stopped while the valve film 152 contacts the bottom surface of a chamber.

Referring to FIGS. 3B and 3C, unlike that the top surface of the valve film 152 of FIG. 3A is flat, the top surfaces of the valve films 152 of FIGS. 3B and 3C are convexed and concaved, respectively.

Aside from the shapes shown in FIGS. 3A through 3C, shape of the valve film 152 may vary. The thickness t of the valve film 152 may be appropriately determined in consideration of a material constituting the second substrate S2 and height of a chamber.

Aside from the structure shown above, the valve unit 150 may have a different structure for stopping flow of a fluid in the channel 130. For example, a thin elastic film and a valve sheet may be arranged in the channel 130. In this case, flow of a fluid may be stopped while the elastic film contacts the valve sheet, whereas the fluid may flow while the elastic film is separated from the valve sheet.

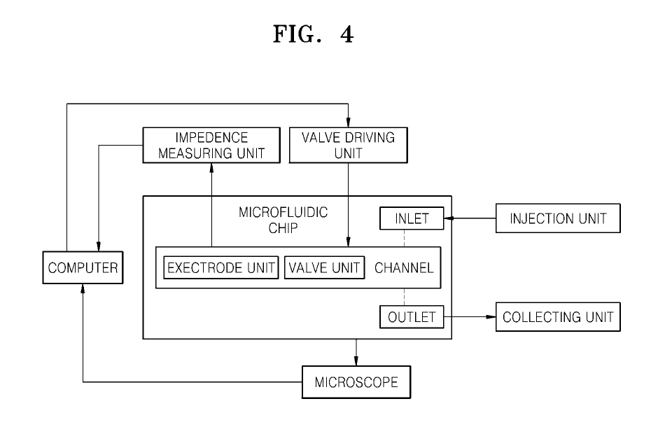

FIG. 4 is a block diagram for describing operation of the cell counter system 100 of FIG. 1.

In FIG. 4, a microfluidic chip refers to a device in which an inlet, a channel, an outlet, a valve unit, and an electrode unit are formed by working a substrate. In other words, FIG. 1 substantially illustrates only a microfluidic chip part of the cell counter system 100.

A fluid containing a plurality of cells is injected by an injection unit to an inlet of a microfluidic chip. The injection unit may be a syringe pump system, for example.

The electrode unit measures impedance according to flow of a fluid, where impedance varies based on whether cells pass the electrode unit. A fluid is a conductive fluid, such as electrolyte, for example, whereas cells contained in the fluid are non-conductive. Therefore, impedance increases when cells pass the electrode unit. The phenomenon appears as a shape of pulse of decreased (reduced) current, and the number of cells contained in the fluid may be counted by counting the pulses. After a desired number of cells is counted, the fluid is drained via an outlet and is collected by a collecting unit. Here, the valve driving unit drives the valve unit to stop flow of the fluid to prevent further flow of cells.

A control unit controls counting based on signals detected by the impedance measuring unit and operation of the valve driving unit. For example, operation of the control unit may be performed by a computer. Furthermore, an image of fluid flow may be captured via a microscope and displayed on a computer monitor.

Accordingly, a number of cells contained in a fluid may be counted precisely and quickly, and, after a desired number of cells is counted, the fluid may be collected by stopping flow of the fluid by driving a valve.

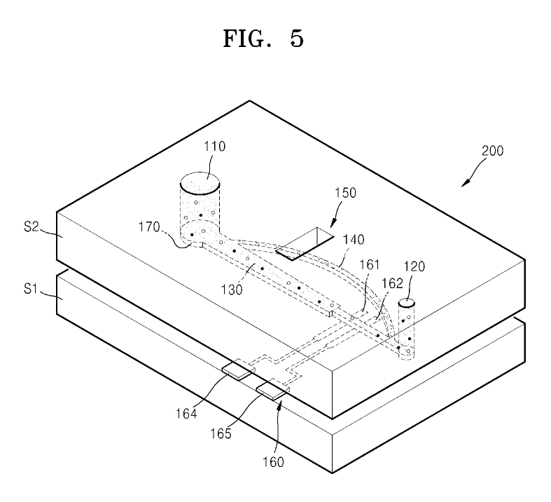

FIG. 5 is a perspective view of a cell counter system 200 according to another embodiment of the present invention.

The cell counter system 200 according to the present embodiment further includes an auxiliary channel 140, which splits at a portion of the channel 130 close to the inlet 110 and is recombined at a portion of the channel 130 close to the outlet 120 and has a width smaller than that of a cell contained in the fluid F.

The auxiliary channel 140 is arranged to help flow of the fluid F which flows from the inlet 110 to the outlet 120. Here, for counting cells, the auxiliary channel 140 is formed to have a width smaller than that of the cells, such that the cells do not pass through the auxiliary channel 140.

The channel 130 and the auxiliary channel 140 may be formed by the spacer 170 sealing a space between the first substrate S1 and the second substrate S2 to have a desired shape. Alternatively, the spacer 170 may be integrated with the first substrate S1 or the second substrate S2.

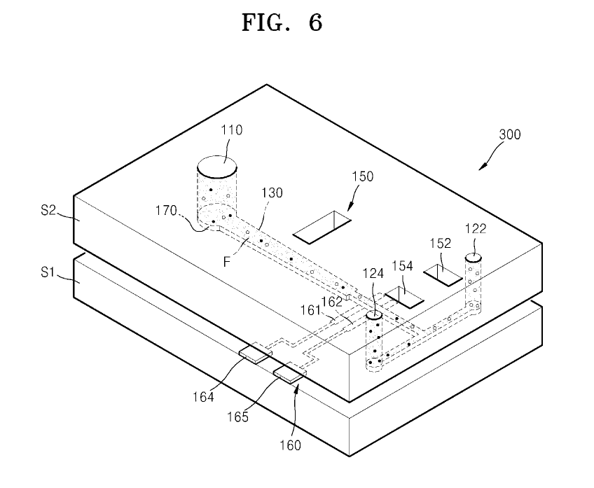

FIG. 6 is a schematic perspective view of a cell counter system 300 according to another embodiment of the present invention.

In the cell counter system 300 according to the present embodiment, the channel 130 is formed to split into two directions at a predetermined location past the electrode unit 160 in a direction in which the fluid F flows. The channel 130 having the above-stated shape may be formed by sealing a space between the first substrate S1 and the second substrate S2 with the spacer 170.

A first outlet 122 and a second outlet 124 are formed at two split ends of the channel 130, where a first auxiliary valve unit 152 for controlling flow of a fluid is arranged between the predetermined location and the first outlet 122, and a second auxiliary valve unit 154 for controlling flow of a fluid is arranged between the predetermined location and the second outlet 124.

Here, a via hole for forming the inlet 110, a via hole for forming the first outlet 122, and a via hole for forming the second outlet 124 are formed in the second substrate S2. Furthermore, grooves for forming the valve unit 150, the first auxiliary valve unit 152, and the second auxiliary valve unit 154 are formed in the second substrate S2. The first auxiliary valve unit 152 and the second auxiliary valve unit 154 may have substantially the same structure as the valve unit 150. Furthermore, the first auxiliary valve unit 152 and the second auxiliary valve unit 154 may have any of structures shown in FIGS. 3A through 3C.

The cell counter system 300 according to the present embodiment is provided for separating cells contained in a fluid assortatively, and collecting different types of cells respectively via different outlets. Here, the cell counter system 300 may further include a control unit (not shown) for categorizing cells into first cells and second cells based on impedances measured by the electrode unit 160. The control unit may also control operation of the first auxiliary valve unit 152 and the second auxiliary valve unit 154, such that the first cells move toward the first outlet 122 and the second cells move toward the second outlet 124.

Therefore, a number of cells contained in the fluid F may be counted assortatively and counted cells may be separated and collected assortatively.

Although FIG. 6 shows assortative counting and collection of two types of cells by using two outlets and two auxiliary valve units, it is merely an example, and a structure capable of assortatively counting and collecting two or more types of cells may be provided by forming two or more outlets and forming an auxiliary valve unit at each channel connected toward each of the outlets.

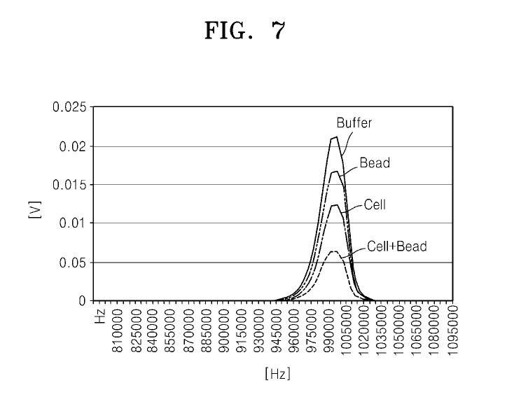

FIG. 7 is a graph of an experiment showing that signals detected by the electrode unit 160 of the cell counter system 300 may differ according to types of cells contained in the fluid F.

The graph shown in FIG. 7 shows changes of voltage when a function generator applies voltage of 10 kHz to electrodes. When only a buffer passes between two electrodes, voltage is about 22 mV. However, when a bead passes between the two electrodes, voltage is dropped to about 17 mV because of a volume occupied by the non-conductive bead. When a cell pass between the two electrodes, since a cell has a relatively larger size, voltage is further dropped to about 10 mV. In a case of a sample containing a cell to which a bead is attached (cell+bead), voltage is further dropped.

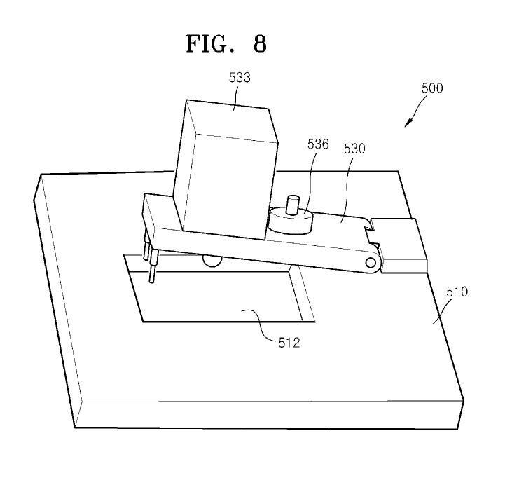

FIG. 8 is a schematic perspective view of a cell counter system 500 according to another embodiment of the present invention.

The cell counter system 500 according to the present embodiment includes a microfluidic chip (not shown), a mount unit 510, a mounting groove 512 formed on the mounting unit 510 for mounting the microfluidic chip, and a cover unit 530, which is hinge-attached, i.e., attached by a hinge, to the mounting unit 510 for opening and closing the mounting groove 512. The cover unit 530 may include an actuator 533 for driving a valve rod and a pumping unit 536 for introducing a fluid via an inlet. The microfluidic chip may be any of the cell counter systems shown in FIGS. 1, 5, and 6, where the present embodiment provides a mechanism for more compact system configuration.

It should be understood that the exemplary embodiments described herein should be considered in a descriptive sense only and not for purposes of limitation. Descriptions of features or aspects within each embodiment should typically be considered as available for other similar features or aspects in other embodiments.

What is claimed is:

1. A cell counter system comprising:
an inlet via which a fluid containing a plurality of cells inflows;
a channel in which the fluid flows;
a valve unit configured to control flow of the fluid in the channel;
an electrode unit, which is arranged in the channel for measuring impedance for counting a number of the plurality of cells; and
an outlet, which is connected to the channel to drain the fluid.

2. The cell counter system of claim 1, further comprising a control unit, which counts a number of cells based on the measured impedance and controls operation of the valve unit.

3. The cell counter system of claim 1, further comprising:
a first substrate and a second substrate, which face each other and are a predetermined distance apart from each other, and
a spacer, which seals a space between the first substrate and the second substrate in a predetermined shape,
wherein the first substrate, second substrate, and spacer together define the channel.

4. The cell counter system of claim 3, wherein the predetermined shape comprises a shape tapered from the inlet to the outlet.

5. The cell counter system of claim 3, wherein
the inlet comprises an inlet via hole,
the outlet comprises an outlet via hole, and
the inlet via hole and the outlet via hole are located in the second substrate.

6. The cell counter system of claim 3, wherein the valve unit comprises:
a valve film, which is a portion of the second substrate having a relatively small thickness; and
a valve rod, which presses a top surface of the valve film, such that the valve film contacts a bottom surface of the channel.

7. The cell counter system of claim 6, wherein the second substrate comprises a first groove having a predetermined depth, and
a bottom surface of the first groove is the top surface of the valve film.

8. The cell counter system of claim 7, wherein the second substrate comprises a second groove on a surface of the second substrate opposite to a surface on which the first groove is located.

9. The cell counter system of claim 7, further comprising:
a mounting unit, on which mounting grooves for mounting the first substrate and the second substrate are located; and
a cover unit, which is attached by a hinge to the mounting unit for opening and closing the mounting grooves and includes an actuator for driving the valve rod and a pumping unit for introducing the fluid via the inlet.

10. The cell counter system of claim 3, wherein the electrode unit comprises a first electrode and a second electrode spaced apart from each other in a direction in which the fluid flows.

11. The cell counter system of claim 3, wherein the electrode unit is arranged on the first substrate.

12. The cell counter system of claim 1, further comprising an auxiliary channel, which splits from the channel at a portion of the channel close to the inlet and is recombined with the channel at a portion of the channel close to the outlet and has a width smaller than that of a cell contained in the fluid.

13. The cell counter system of claim 12, further comprising:
a first substrate and a second substrate, which face each other, are a predetermined distance apart from each other, and define the channel and the auxiliary channel; and
a spacer, which seals a space between the first substrate and the second substrate in a predetermined shape.

14. The cell counter system of claim 1, wherein the channel is split into two directions at a predetermined location past the electrode unit in a direction in which the fluid flows.

15. The cell counter system of claim 14, wherein the outlet comprises a first outlet and a second outlet that are respectively located at ends of the channel split into two directions.

16. The cell counter system of claim 15, further comprising:
a first substrate and a second substrate, which face each other, are a predetermined distance apart from each other, and define the channel;
and a spacer, which seals a space between the first substrate and the second substrate in a predetermined shape.

17. The cell counter system of claim 16, wherein
the inlet comprises an inlet via hole,
the first outlet comprises a first outlet via hole,
the second outlet comprises a second outlet via hole, and
the inlet via hole, the first outlet via hole, and the second outlet via hole are located in the second substrate.

18. The cell counter system of claim 17, further comprising:
a first auxiliary valve unit for controlling flow of a fluid located between the predetermined location and the first outlet; and
a second auxiliary valve unit for controlling flow of a fluid located between the predetermined location and the second outlet.

19. The cell counter system of claim 18, wherein each of the first and second auxiliary valve units comprise:
a valve film, which is a portion of the second substrate having a relatively small thickness; and
a valve rod, which presses a top surface of the valve film, such that the valve film contacts a bottom surface of the channel.

20. The cell counter system of claim 19, wherein the second substrate comprises grooves, each groove having a predetermined depth and corresponding to one of the valve unit, the first auxiliary valve unit, and the second auxiliary valve unit.

21. The cell counter system of claim 20, further comprising:
a mounting unit, on which mounting grooves for mounting the first substrate and the second substrate are located; and
a cover unit, which is attached by a hinge to the mounting unit for opening and closing the mounting grooves and includes an actuator for driving the valve rods and a pumping unit for introducing the fluid via the inlet.

22. The cell counter system of claim 18, further comprising a control unit, which categorizes cells into first cells and second cells based on measured impedance and controls operation of the first auxiliary valve unit and the second auxiliary valve unit, such that the first cells move toward the first outlet and the second cells move toward the second outlet.
